(19) United States
(12) Patent Application Publication
(10) Pub. No.: US 2022/0378376 A1
(43) Pub. Date: Dec. 1, 2022

(54) WEARABLE ELECTRONIC DEVICE AND BIOLOGICAL INFORMATION MEASURING SYSTEM CAPABLE OF SENSING MOTION OR CALIBRATING BIOLOGICAL INFORMATION CORRESPONDING TO MOTION

(71) Applicant: PixArt Imaging Inc., Hsin-chu City (TW)

(72) Inventor: Ren-Hau Gu, Hsin-Chu City (TW)

(21) Appl. No.: 17/331,616

(22) Filed: May 26, 2021

(51) Int. Cl. A61B 5/00

(52) U.S. Cl. CPC A61B 5/721; A61B 5/6843; A61B 5/0059; A61B 5/681; A61B 2562/182

(57) ABSTRACT

A wearable electronic device, comprising: a substrate: a first motion sensing region, comprising at least one first electrode on the substrate; a second motion sensing region, comprising at least one second electrode, wherein a shielding layer is provided on the second electrode, and the second electrode is between the shielding layer and the substrate, wherein a user causes more capacitance variation to the first electrodes and causes less capacitance variation to the second electrodes when the user wears the smart watch; a capacitance calculating circuit, coupled to the first electrode, configured to calculate a capacitance variation generated by the first electrode or the second electrode; and a motion determination circuit, configured to determine a motion of the wearable electronic device according to the capacitance variation of the first electrode or the capacitance variation of the second electrode.

### BACKGROUND OF THE INVENTION

### 1. Field of the Invention

The present invention relates to a wearable electronic device and a biological information measuring system, and particularly relates to a wearable electronic device and a biological information measuring system capable of sensing motion or calibrating biological information measuring corresponding to motion.

### 2. Description of the Prior Art

In recent years, a smart wearable electronic device such as a smart watch or a smart wristband has become more and more popular. Such smart wearable electronic device always has the function of biological information measuring (e.g. blood pressure, heart rate). The motion of the user (e.g., jogging, walking . . . ) may cause interference to biological information measuring when the user wears the smart wearable electronic device. However, a conventional smart wearable electronic device has no proper mechanism for calibrating the biological information measuring.

Also, a conventional smart wearable electronic device is hard to precisely determine the motion.

### SUMMARY OF THE INVENTION

Therefore, one objective of the present invention is to provide a wearable electronic device which can sense motion or calibrates biological information corresponding to the motion.

Another objective of the present invention is to provide a biological information measuring system which can sense motion and calibrates biological information corresponding to the motion.

One embodiment of the present invention discloses a wearable electronic device, comprising: a substrate: a first motion sensing region, comprising at least one first electrode on the substrate; a second motion sensing region, comprising at least one second electrode, wherein a shielding layer is provided on the second electrode, and the second electrode is between the shielding layer and the substrate, wherein a user causes more capacitance variation to the first electrodes and causes less capacitance variation to the second electrodes when the user wears the smart watch; a capacitance calculating circuit, coupled to the first electrode, configured to calculate a capacitance variation generated by the first electrode or the second electrode; and a motion determination circuit, configured to determine a motion of the wearable electronic device according to the capacitance variation of the first electrode or the capacitance variation of the second electrode.

Another embodiment of the present invention discloses a biological information measuring system comprising: a biological information measuring device, configured to measure biological information; a substrate; a first motion sensing region, comprising at least one first electrode on the substrate; a second motion sensing region, comprising at least one second electrode, wherein a shielding layer is provided on the second electrode, and the second electrode is between the shielding layer and the substrate, wherein a user causes more capacitance variation to the first electrodes and causes less capacitance variation to the second electrodes when the user wears the smart watch; a capacitance calculating circuit, coupled to the first electrode, configured to calculate a capacitance variation generated by the first electrode or the second electrode; and a motion determination circuit, configured to determine a motion of the biological information measuring system according to the capacitance variation of the first electrode or the capacitance variation of the second electrode. The biological information measuring system calibrates the biological information corresponding to the motion.

In view of above-mentioned embodiments, the biological information can be calibrated corresponding to motions, thus the biological information measuring can be more accurate.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are schematic diagrams illustrating a smart watch according to one embodiment of the present invention.

FIG. 3 is a cross section view of the smart watch illustrated in FIG. 2.

FIG. 4 is a block diagram illustrating a smart watch according to one embodiment of the present invention.

FIG. 5 is a schematic diagram illustrating a smart watch according to another embodiment of the present invention.

FIG. 6 and FIG. 7 are schematic diagram illustrating biological information measuring devices according to embodiments of the present invention.

### DETAILED DESCRIPTION

In following descriptions, a plurality of embodiments are provided to explain the concepts of the present invention. Please note the components in following embodiments can be implemented by hardware (e.g. circuit or device), or implemented by firmware (e.g. a processor installed with at least one program). Also, the components in each embodiment can be integrated to fewer components, or be divided to more components.

Additionally, in following embodiments, a smart watch is taken as an example to explain the concepts of the present invention. However, the wearable electronic device is not limited to a smart watch, it can be any other wearable electronic device, such as a smart wristband.

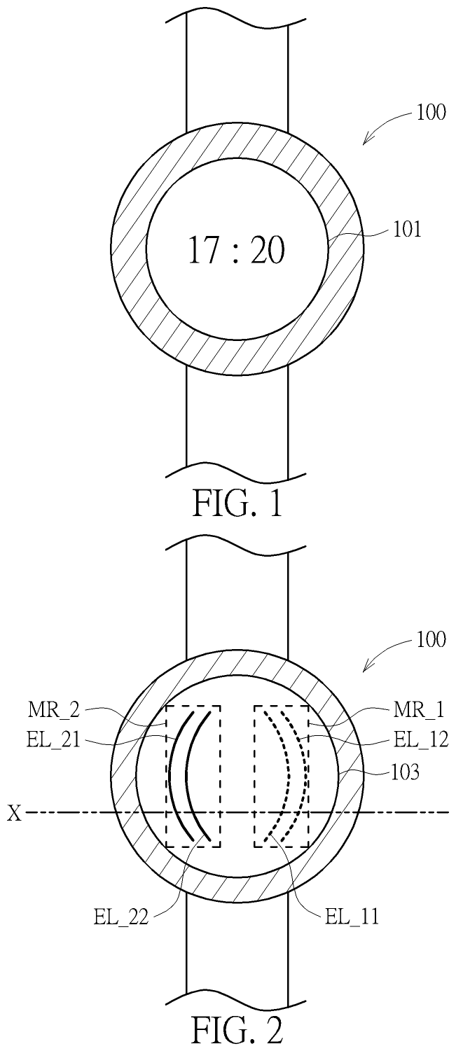

FIG. 1 and FIG. 2 are schematic diagrams illustrating a smart watch 100 according to one embodiment of the present invention. As illustrated in FIG. 1, the smart watch 100 comprises a front surface 101 which can show desired information such as time, messages, or images, such as a display. In FIG. 2, the smart watch 100 comprises a sensing surface 103 which is a back surface of the smart watch 100 in this embodiment, by which a user can cause capacitance variation to the electrodes included in the smart watch 100 when the user wears the smart watch 100. However, the sensing surface 103 can be any other surface of an electronic device if the concept of the present invention is applied to another type of electronic device.

As illustrated in FIG. 2, the smart watch 100 comprises a first motion sensing region MR_1 and a second motion sensing region MR_2. The first motion sensing region MR_1 comprises at least one first electrode (only two first electrodes EL_11, EL_12 are illustrated). Also, the second motion sensing region MR_2 comprises at least one second electrode (only two second electrodes EL_21, EL_22 are illustrated). A user cause more capacitance variation to the first electrodes EL_11, EL_12 and causes less capacitance variation to the second electrodes EL_21, EL_22, when the user wears the smart watch 100. In other words, the capacitance variation which the user causes to the second electrodes EL_21, EL_22 while wearing the smart watch 100 is less than the capacitance variation which the user causes to the first electrodes EL_11, EL_12 while wearing the smart watch 100. In one embodiment, a shielding layer is provided on the second electrodes EL_21, EL_22 such that the user causes less capacitance variation to the second electrodes EL_21, EL_22 while wearing the smart watch 100. In one embodiment, the shielding layer can completely isolate the second electrodes EL_21, EL_22 and user's skins, such that the user does not cause any capacitance variation to the second electrodes EL_21, EL_22 while wearing the smart watch 100.

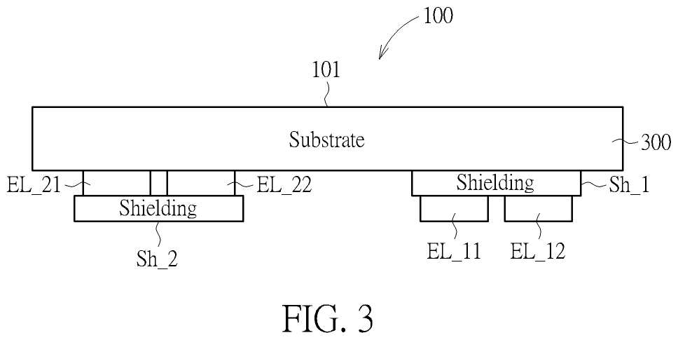

FIG. 3 is a cross section view of the smart watch illustrated in FIG. 2. Specifically, FIG. 3 is a cross section view of the smart watch 100 illustrated in FIG. 2, following the dotted line X. As illustrated in FIG. 3, the smart watch 100 comprises a substrate 300. The substrate 300 can be, for example, a case in which the circuits of smart watch 100 is provided. Alternatively, the substrate 300 can be a substrate provided to the case in which the circuits of smart watch 100 is provided. The first electrodes EL_11 and EL_12 are provided in the substrate 300 and a portion of the first electrode EL_11 and EL_12 expose outside the substrate 300. Also, a first shielding layer Sh_1 is provided between the substrate 300 and the first electrodes EL_11, EL_12.

Additionally, the second electrodes EL_21, EL_22 are also provided in the substrate 300 and a portion of the second electrodes EL_21, EL_22 expose outside the substrate 300. However, different from the first electrodes EL_11, EL_12, the second electrodes EL_21, EL_22 are between the second shielding layer Sh_2 and the substrate 300. In one embodiment, the first shielding layer Sh_1 and the second shielding layer Sh_2 are coupled to a ground voltage level in the smart watch 100. Also, in one embodiment, the first shielding layer Sh_1 and the second shielding layer Sh_2 are metal layers.

The first electrodes EL_11, EL_12 and the second electrodes EL_21, EL_22 can be mutual capacitance electrodes or self-capacitance electrodes. If the electrode is a mutual capacitance electrode, the electrode only serves as one of a transmitter (TX) and a receiver (RX). If the electrode is a self-capacitance capacitance electrode, it serves as a transmitter (TX) and a receiver (RX). In one embodiment, the first electrode EL_11 and the second electrode EL_21 are mutual capacitance electrodes which serve as transmitters, and the first electrode EL_12 and the second electrode EL_22 are mutual capacitance electrodes which serve as receivers. In such case, the first electrode EL_11 and the second electrode EL_21 can be coupled to the same signal source which generates a signal for detecting a touch, but not limited.

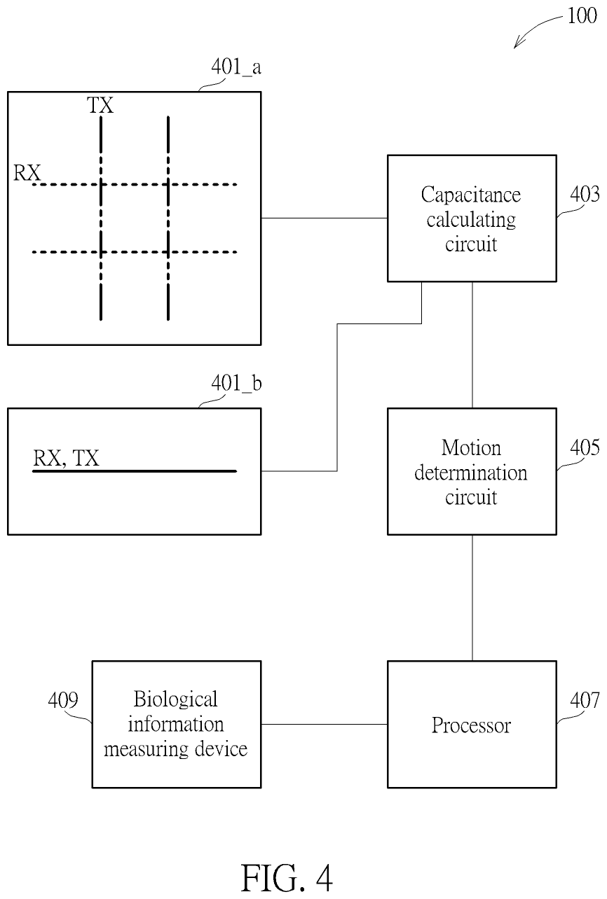

FIG. 4 is a block diagram illustrating a smart watch 100 according to one embodiment of the present invention. Specifically, FIG. 4 illustrates how the capacitance variation of the first electrodes EL_11, EL_12 and the second electrodes EL_21, EL_22 are calculated and how the motion is determined.

As illustrated in FIG. 4, the smart watch 100 comprises at least one electrode (401_a or 401_b), a capacitance calculating circuit 403 and a motion determination circuit 405. The electrode can be any one or a plurality of the first electrodes EL_11, EL_12 and the second electrodes EL_21, EL_22. The capacitance calculating circuit 403 is configured to calculate a capacitance variation generated by at least one electrode (401_a or 401_b). The motion determination circuit 405 is configured to determine the motion according to the capacitance variation. The motion can be any user's actions, such as lying down, sitting, walking, running or jogging.

The electrode in FIG. 4 can be the mutual capacitance electrode 401_a or the self-capacitance electrode 401_b. The mutual capacitance electrode 401_a means one electrode only serves one of a transmitter (TX) and a receiver (RX). Also, the self-capacitance electrode 401_b means a single electrode serves as a transmitter (TX) and a receiver (RX). Therefore, if the electrode is the mutual capacitance electrode 401_a, the capacitance calculating circuit 403 calculates capacitance variation between different electrodes. Further, if the electrode is the self-capacitance electrode 401_b, the capacitance calculating circuit 403 calculates capacitance variation of a single electrode. Details of the mutual capacitance electrode 401_a and the self-capacitance electrode 401_b are well known by persons skilled in the art, thus are omitted for brevity here.

In one embodiment, the smart watch 100 further comprises a processor 407 and a biological information measuring device 409 which can measure biological information such as but not limited to heart rate, blood pressure, blood oxygen concentration . . . . In such case, after the motion determination circuit 405 determines the motion, the processor 407 calibrates the biological information measured by the biological information measuring device 409 corresponding to the motion. It will be appreciated that the processor 407 can be integrated to any other component of the smart watch 100.

Many algorithms can be applied to calibrate the biological information. For example, different motions may correspond to different noise waves in frequency domain or noise values, thus the biological information can be calibrated based on the noise waves or noise values. For another example, a particular motion may cause more noises in some frequency bands. In such case, waves of the biological information in the frequency bands which have more noises can be removed, thereby the biological information can be calibrated corresponding to the motion.

In one embodiment, different motion sensing regions are respectively sensitive to different motions. In such case, the motion determining circuit 405 determines different motions based on capacitance variation of different motion sensing regions. For example, the motion determination circuit 405 determines whether a first motion exists according to the capacitance variation of the first electrode EL_11, EL_12 and determines whether a second motion exists according to the capacitance variation of the second electrode EL_21, EL_22. In one embodiment, the first motion comprises walking and the second motion comprises running or jogging, but not limited. Therefore, comparing with the conventional smart watch, the smart watch 100 illustrated in FIG. 2 has the advantage of “different motion sensing regions are respectively sensitive to different motions”.

In such case, the processor 407 calibrates the biological information corresponding to the first motion when the motion determination circuit 405 determines the first motion exists, and calibrates the biological information corresponding to the second motion when the motion determination circuit 405 determines the second motion exists.

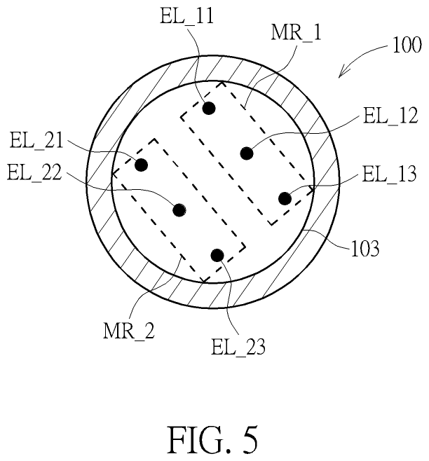

The arrangements and/or the numbers of the motion sensing regions and the electrodes in the motion sensing regions are not limited to the example illustrated in FIG. 2. FIG. 5 is a schematic diagram illustrating a smart watch according to another embodiment of the present invention. As illustrated in FIG. 5, the locations of the first motion sensing region MR_1 and the second motion sensing region MR_2 are different from which in FIG. 2. Also, the first motion sensing region MR_1 in FIG. 5 comprises three first electrodes EL_11, EL_12 and EL_13 rather than two first electrodes EL_11, EL_12 shown in FIG. 2. Additionally, the second motion sensing region MR_2 in FIG. 5 comprises three second electrodes EL_21, EL_22 and EL_23 rather than two second electrodes EL_21, EL_22 shown in FIG. 2. Furthermore, the first electrodes EL_11, EL_12 and EL_13 and the second electrodes EL_21, EL_22 and EL_23 in FIG. 5 are round rather than the curved-line shape illustrated in FIG. 2. Therefore, the arrangements and/or the numbers of the motion sensing regions and the electrodes in the motion sensing regions can be changed corresponding to different requirements. Such variation should also fall in the scope of the present invention.

As above-mentioned description, the biological information measuring device 409 can measure biological information such as heart rate, blood pressure, blood oxygen concentration. The biological information measuring device 409 may have various structures. In one embodiment, the biological information measuring device 409 measures biological information based on optical data such as images or any other optical data having optical features.

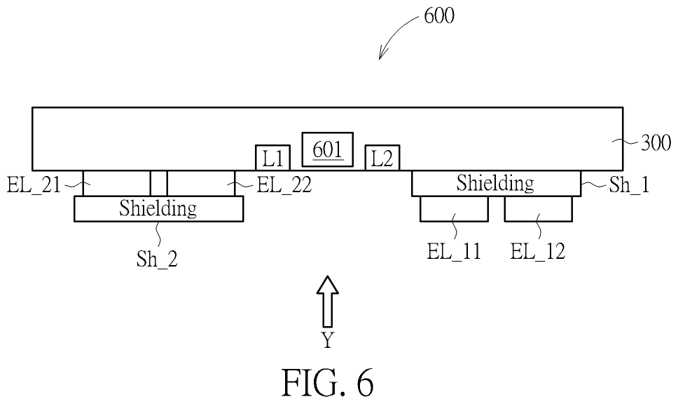

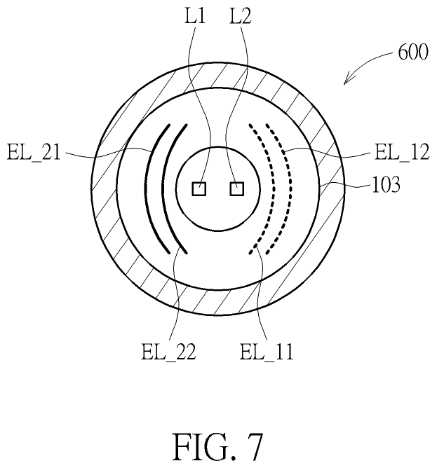

FIG. 6 and FIG. 7 are schematic diagrams illustrating biological information measuring devices according to embodiments of the present invention. As illustrated in FIG. 6, the biological information measuring device in the smart watch 600 comprises an optical sensor 601 and light sources L1, L2. The light sources L1, L2 are configured to generate light. The optical sensor 601 is configured to sense optical data generated according to the light. The optical data is used for computing the biological information. The smart watch 600 illustrated in FIG. 6 further comprises the first electrode EL_12 and the second electrode EL_22 illustrated in FIG. 3. That is, the smart watch 600 can be combined with the embodiments illustrated in FIG. 2 and FIG. 3. However, the smart watch 600 can comprise motion sensing regions having electrodes with different shapes.

FIG. 7 is a schematic diagram illustrating a structure view in the Y direction of FIG. 6. As shown in FIG. 7, the light sources L1, L2 are disposed on the sensing surface 103 and are surrounded by the first electrodes EL_11, EL_12 and the second electrodes EL_21, EL_22. However, the arrangements and/or the numbers of the optical sensor and the light source of the biological information measuring device are not limited to the embodiments illustrated in FIG. 6 and FIG. 7.

Please note, the smart watch provided by the present invention is not limited to comprise the biological information measuring device. Also, if the smart watch comprises the information measuring device, the smart watch can be regarded as a biological information measuring system.

In view of above-mentioned embodiments, the biological information can be calibrated corresponding to motions, thus the biological information measuring can be more accurate. Also, the motion can be precisely determined via different motion sensing regions.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A wearable electronic device, comprising:
a substrate:
a first motion sensing region, comprising at least one first electrode on the substrate;
a second motion sensing region, comprising at least one second electrode, wherein a shielding layer is provided on the second electrode, and the second electrode is between the shielding layer and the substrate, wherein a user causes more capacitance variation to the first electrodes and causes less capacitance variation to the second electrodes when the user wears the smart watch;
a capacitance calculating circuit, coupled to the first electrode, configured to calculate a capacitance variation generated by the first electrode or the second electrode; and
a motion determination circuit, configured to determine a motion of the wearable electronic device according to the capacitance variation of the first electrode or the capacitance variation of the second electrode.

2. The wearable electronic device of claim 1, further comprising:
a biological information measuring device, configured to measure biological information of an user;
wherein the wearable electronic device calibrates the biological information corresponding to the motion.

3. The wearable electronic device of claim 2, wherein the biological information measuring device comprises:
at least one light source, configured to generate light; and
an optical sensor, configured to sense optical data generated according to the light;
wherein the optical data is used for computing the biological information.

4. The wearable electronic device of claim 1, wherein the motion determination circuit determines whether a first motion exists according to the capacitance variation of the first electrode and determines whether a second motion exists according to the capacitance variation of the second electrode.

5. The wearable electronic device of claim 4, further comprising:
a biological information measuring device, configured to measure a biological information of an user;
wherein the wearable electronic device calibrates the biological information corresponding to the first motion when the motion determination circuit determines the first motion exists, and calibrates the biological information corresponding to the second motion when the motion determination circuit determines the second motion exists.

6. The wearable electronic device of claim 5, wherein the biological information measuring device comprises:
at least one light source, configured to generate light; and
an optical sensor, configured to sense optical data generated according to the light;
wherein the optical data is used for computing the biological information.

7. The wearable electronic device of claim 1, wherein the shielding layer is coupled to a ground voltage level of the wearable electronic device.

8. The wearable electronic device of claim 1, wherein the shielding layer is a metal layer.

9. The wearable electronic device of claim 1, further comprising a sensing surface, by which a user can cause the capacitance variation to the first electrode when the user wears the wearable electronic device.

10. The wearable electronic device of claim 8, wherein the wearable electronic device comprises a front surface which can show desired information, and comprises a back surface served as the sensing surface.

11. A biological information measuring system, comprising:
a biological information measuring device, configured to measure biological information;
a substrate;
a first motion sensing region, comprising at least one first electrode on the substrate;
a second motion sensing region, comprising at least one second electrode, wherein a shielding layer is provided on the second electrode, and the second electrode is between the shielding layer and the substrate, wherein a user causes more capacitance variation to the first electrodes and causes less capacitance variation to the second electrodes when the user wears the smart watch;
a capacitance calculating circuit, coupled to the first electrode, configured to calculate a capacitance variation generated by the first electrode or the second electrode; and
a motion determination circuit, configured to determine a motion of the biological information measuring system according to the capacitance variation of the first electrode or the capacitance variation of the second electrode;
wherein the biological information measuring system calibrates the biological information corresponding to the motion.

12. The biological information measuring system of claim 11, wherein the biological information measuring device comprises:
a light source, configured to generate light; and
an optical sensor, configured to sense optical data generated according to the light;
wherein the optical data is used for computing the biological information.

13. The biological information measuring system of claim 11, wherein the motion determination circuit determines whether a first motion exists according to the capacitance variation of the first electrode and determines whether a second motion exists according to the capacitance variation of the second electrode.

14. The biological information measuring system of claim 13, wherein the biological information measuring system calibrates the biological information corresponding to the first motion when the motion determination circuit determines the first motion exists, and calibrates the biological information corresponding to the second motion when the motion determination circuit determines the second motion exists.

15. The biological information measuring system of claim 11, wherein the shielding layer is coupled to a ground voltage level of the biological information measuring system.

16. The biological information measuring system of claim 11, wherein the shielding layer is a metal layer.
